(19) United States
(12) Patent Application Publication
(10) Pub. No.: US 2004/0240897 A1
(43) Pub. Date: Dec. 2, 2004

(54) Liquid toner screening device

(75) Inventors: Hsin Hsin Chou, Woodbury, MN (US); Truman Frank Kellie, Lakeland, MN (US); William D. Edwards, New Richmond, WI (US); Brian P. Teschendorf, Vadnais Heights, MN (US)

Correspondence Address: Mark A. Litman & Associates, P. A., York Business Center, Suite 205, 3209 West 76th St., Edina, MN, 55435, US

(73) Assignee: SAMSUNG Electronics Co. Ltd

(21) Appl. No.: 10/448,577

(22) Filed: May 30, 2003

(51) Int. Cl. G03G 21/00; G01N 33/00

(52) U.S. Cl. 399/29; 399/15; 73/866

(57) ABSTRACT

A prospective liquid toner is evaluated for potential electrostatic imaging performance. A drop of the prospective toner is applied on a flat surface, the prospective toner is electrically plated onto an electrically resistive compliant roller. The roller carries the plated liquid toner and applied the plated toner to a substrate. The plated toner is transferred to the substrate and the transferred toner qualities such as least length, width, and shape are compared to standards expected from a liquid toner of acceptable performance characteristics.

### BACKGROUND OF THE INVENTION

1. Field of the Invention

This invention relates to the field of liquid electrophotography, and specifically to a method and apparatus for screening liquid toners for use in electrophotographic printing devices.

2. Background of the Art

In electrophotographic and electrostatic and imaging processes (collectively electrographic processes), an electrostatic image is formed on the surface of a photoreceptive element or dielectric element, respectively. The photoreceptive element or dielectric element may be an intermediate transfer sheet, drum or belt or the substrate for the final toned image itself, as described by Schmidt, S. P. and Larson, J. R. in Handbook of Imaging Materials, Diamond, A. S., Ed: Marcel Dekker: New York; Chapter 6, pp 227-252, and U.S. Pat. Nos. 4,728,983; 4,321,404; and 4,268,598.

In electrostatic printing, a latent image is typically formed by (1) placing a charge image onto a dielectric element (typically the receiving substrate) in selected areas of the element with an electrostatic writing stylus or its equivalent to form a latent charge image. This latent charge image is developed or toned by (2) applying toner to the charge image, and (3) fixing the toned image. An example of this type of process is described in U.S. Pat. No. 5,262,259.

In electrophotographic printing, also referred to as xerography, electrophotographic technology is used to produce images on a final image receptor, such as paper, film, drums, or the like. Electrophotographic technology is incorporated into a wide range of equipment including photocopiers, laser printers, facsimile machines, and the like.

Electrophotography typically involves the use of a reusable, light sensitive, temporary charge accepting, temporary image receptor, known as a photoreceptor. The photoreceptor is used in the process of producing an electrophotographic image on a final, permanent image receptor. A representative electrophotographic process involves a series of steps to produce a visible toned image on a receptor, including charging of the photoreceptor, exposure to dissipate the charge in an imagewise manner and form a latent charge image, toner development of the latent charge image, transfer of the toned image, fusing of the transferred toned image, cleaning of the photoreceptor, and erasure of residual charge on the photoreceptor.

In the charging step, a photoreceptor is covered with charge of a desired polarity, either negative or positive, typically with a corona device or charging roller. In the exposure step, an optical system, typically a laser scanner or diode array, forms a latent charge image by selectively discharging the charged surface of the photoreceptor in an imagewise manner corresponding to the desired image to be formed on the final image receptor. In the development step, toner particles of the appropriate polarity are generally brought into contact with the latent charge image on the photoreceptor, typically using a developer that is electrically-biased to a potential opposite in polarity to the toner polarity. The toner particles migrate to the photoreceptor and selectively adhere to the latent charge image via electrostatic forces, forming a temporary toned image on the photoreceptor.

In the transfer step, the temporary toned image is transferred from the photoreceptor to the desired final image receptor. An intermediate transfer element is sometimes used to effect transfer of the toned image (usually to accomplish a desired order of color planes in the image) from the photoreceptor with subsequent transfer of the toned image to a final image receptor. In the fusing step, the toned image on the final image receptor is heated to soften or melt the toner particles, thereby fusing the toned image to the final receptor to form a final and permanent image. An alternative fusing method involves fixing the toner to the final receptor under high pressure with or without heat. In the cleaning step, residual toner remaining on the photoreceptor is removed.

Finally, in the erasing step, the photoreceptor charge is reduced to a substantially uniformly low value by exposure to light of a particular wavelength band, thereby removing remnants of the original latent image and preparing the photoreceptor for the next imaging cycle.

Two types of toner are in widespread, commercial use: liquid toner and dry toner. The term “dry” does not mean that the dry toner is totally free of any liquid constituents, but connotes that the toner particles do not contain any significant amount of solvent (or gives the toner a liquid appearance), e.g., typically less than 10 weight percent solvent and preferably less then 8% or less then 5% by total weight of toner (generally, dry toner is as dry as is reasonably practical in terms of solvent content), and the dry toner particles are capable of carrying a triboelectric charge. This relative proportion of liquid carrier is a physical characteristic that distinguishes dry toner particles from liquid toner particles.

A typical liquid toner composition generally includes toner particles suspended or dispersed in a liquid carrier. The liquid carrier is typically a nonconductive dispersant liquid, the lack of charge carrying capability being necessary to avoid discharging the latent electrostatic image. Liquid toner particles are generally solvated or stabilized (dispersed and suspended) to some degree in the liquid carrier (or carrier liquid), typically in more than 50 weight percent (by total weight of the toner) of a low polarity, low dielectric constant, substantially nonaqueous carrier solvent. Liquid toner particles are generally chemically charged using polar groups that dissociate in the carrier solvent, but the toner particles do not carry a triboelectric charge while solvated and/or dispersed in the liquid carrier. Liquid toner particles are also typically smaller than dry toner particles. Because of their small particle size, ranging from about 5 microns to sub-micron size, liquid toners are capable of producing very high-resolution toned images.

A typical toner particle for a liquid toner composition generally comprises a visual enhancement additive (for example, a colored pigment particle) and a polymeric binder. The polymeric binder fulfills functions both during and after the electrophotographic process, supporting the visual enhancement additive during toning and fusing the visual enhancement additive during formation of the permanent image. With respect to processability, the character of the binder impacts charging and charge stability, flow, and fusing characteristics of the toner particles. These characteristics are important to achieve good performance during development, transfer, and fusing. After an image is formed on the final receptor, the nature of the binder (e.g., glass transition temperature, melt viscosity, molecular weight) and the fusing conditions (e.g., temperature, pressure and fuser configuration) impact the durability (e.g., blocking and erasure resistance), adhesion to the receptor, gloss, and the like.

Polymeric binder materials suitable for use in liquid toner particles typically exhibit glass transition temperatures of from about −24° C. to 55° C., which is lower than the range of glass transition temperatures (50-100° C.) typical for polymeric binders used in dry toner particles. In particular, some liquid toners are known to incorporate polymeric binders exhibiting glass transition temperatures (Tg) below room temperature (25° C.) to rapidly self fix, e.g., by film formation, in the liquid electrophotographic imaging process; see e.g., U.S. Pat. No. 6,255,363. However, such liquid toners are also known to exhibit inferior image durability (e.g., poor blocking properties and erasure resistance) resulting from the low T_{g} after fusing the toned image to a final image receptor.

In other printing processes using liquid toners, self-fixing is not required. In such a system, the image developed on the photoconductive surface is transferred to an intermediate transfer belt (“ITB”) or intermediate transfer member (“ITM”) or directly to a print medium without film formation at this stage. See, for example, U.S. Pat. No. 5,410,392 to Landa, issued on Apr. 25, 1995; and U.S. Pat. No. 5,115,277 to Camis, issued on May 19, 1992. In such a system, this transfer of discrete toner particles in image form is carried out using a combination of mechanical forces, electrostatic forces, and thermal energy. In the system particularly described in the U.S. Pat. No. 5,115,277 Camis patent, DC bias voltage is connected to an inner sleeve member to develop electrostatic forces at the surface of the print medium for assisting in the efficient transfer of color images.

The toner particles used in such a system have been previously prepared using conventional polymeric binder materials, and not polymers made using an organosol process. Thus, for example the U.S. Pat. No. 5,410,392 Landa patent states that the liquid developer to be used in the disclosed system is described in U.S. Pat. No. 4,794,651 (also to Landa), issued on Dec. 27, 1988. This former Landa patent discloses liquid toners made by heating a preformed high T_{g} polymer resin in a carrier liquid to an elevated temperature sufficiently high for the carrier liquid to soften or plasticize the resin, adding a pigment, and exposing the resulting high temperature dispersion to a high energy mixing or milling process.

Although such non self-fixing liquid toners using higher T_{g} (T_{g} generally greater than or equal to about 60° C.) polymeric binders should have good image durability, such toners are known to exhibit other problems related to the choice of polymeric binder, including image defects due to the inability of the liquid toner to rapidly self fix in the imaging process, poor charging and charge stability, poor stability with respect to agglomeration or aggregation in storage, poor sedimentation stability in storage, and the requirement that high fusing temperatures of about 200-250° C. be used in order to soften or melt the toner particles and thereby adequately fuse the toner to the final image receptor.

To overcome the durability deficiencies, polymeric materials selected for use in both nonfilm-forming liquid toners and dry toners more typically exhibit a range of T_{g} of at least about 55-65° C. to obtain good blocking resistance after fusing, yet typically require high fusing temperatures of about 200-250° C. to soften or melt the toner particles and thereby adequately fuse the toner to the final image receptor. High fusing temperatures are a disadvantage for dry toners because of the long warm-up time and higher energy consumption associated with high temperature fusing and because of the risk of fire associated with fusing toner to paper at temperatures approximating or approaching the autoignition temperature of paper (233° C.).

In addition, some liquid and dry toners using high T_{g} polymeric binders are known to exhibit undesirable partial transfer (offset) of the toned image from the final image receptor to the fuser surface at temperatures above or below the optimal fusing temperature, requiring the use of low surface energy materials in the fuser surface or the application of fuser oils to prevent offset. Alternatively, various lubricants or waxes have been physically blended into the dry toner particles during fabrication to act as release or slip agents; however, because these waxes are not chemically bonded to the polymeric binder, they may adversely affect triboelectric charging of the toner particle or may migrate from the toner particle and contaminate the photoreceptor, an intermediate transfer element, the fuser element, or other surfaces critical to the electrophotographic process.

In addition to the polymeric binder and the visual enhancement additive, liquid toner compositions can optionally include other additives. For example, charge control agents can be added to impart an electrostatic charge on the toner particles. Dispersing agents can be added to provide colloidal stability, to aid fixing of the image, and to provide charged or charging sites for the particle surface. Dispersing agents are commonly added to liquid toner compositions because toner particle concentrations are high (inter-particle distances are small) and electrical double-layer effects alone will not adequately stabilize the dispersion with respect to aggregation or agglomeration. Release agents can also be used in the toner to help prevent the toner from sticking to fuser rolls when those are used. Other additives include antioxidants, ultraviolet stabilizers, antistatic agents, fungicides, bactericides, flow control agents, and the like.

One fabrication technique used in the manufacture of toners involves synthesizing an amphipathic copolymeric binder dispersed in a liquid carrier to form an organosol, then mixing the formed organosol with other ingredients to form a liquid toner composition. Typically, organosols are synthesized by nonaqueous dispersion polymerization of polymerizable compounds (e.g., monomers) to form copolymeric binder particles that are dispersed in a low dielectric hydrocarbon solvent (carrier liquid). These dispersed copolymer particles are sterically-stabilized with respect to aggregation by chemical bonding of a steric stabilizer (e.g., graft stabilizer), solvated by the carrier liquid, to the dispersed core particles as they are formed in the polymerization. Details of the mechanism of such steric stabilization are described in Napper, D. H., “Polymeric Stabilization of Colloidal Dispersions,” Academic Press, New York, N.Y., 1983. Procedures for synthesizing self-stable organosols are described in “Dispersion Polymerization in Organic Media,” K. E. J. Barrett, ed., John Wiley: New York, N.Y., 1975.

Liquid toner compositions have been manufactured using dispersion polymerization in low polarity, low dielectric constant carrier solvents for use in making relatively low glass transition temperature (T_{g}≦30° C.) film-forming liquid toners that undergo rapid self-fixing in the electrophotographic imaging process. See, for example, U.S. Pat. Nos. 5,886,067 and 6,103,781. Organosols have also been prepared for use in making intermediate glass transition temperature (T_{g} between 30-55° C.) liquid electrostatic toners for use in electrostatic stylus printers. See, for example, U.S. Pat. No. 6,255,363 B1. A representative non-aqueous dispersion polymerization method for forming an organosol is a free radical polymerization carried out when one or more ethylenically-unsaturated monomers, soluble in a hydrocarbon medium, are polymerized in the presence of a preformed, polymerizable solution polymer (e.g. a graft stabilizer or “living” polymer). See U.S. Pat. No. 6,255,363.

Once the organosol has been formed, one or more additives can be incorporated, as desired. For example, one or more visual enhancement additives and/or charge control agents can be incorporated. The composition can then subjected to one or more mixing processes, such as homogenization, microfluidization, ball-milling, attritor milling, high energy bead (sand) milling, basket milling or other techniques known in the art to reduce particle size in a dispersion. The mixing process acts to break down aggregated visual enhancement additive particles, when present, into primary particles (having a diameter in the range of about 0.05 to 1.0 microns) and may also partially shred the dispersed copolymeric binder into fragments that can associate with the surface of the visual enhancement additive.

According to this embodiment, the dispersed copolymer or fragments derived from the copolymer then associate with the visual enhancement additive, for example, by adsorbing to or adhering to the surface of the visual enhancement additive, thereby forming toner particles. The result is a sterically-stabilized, nonaqueous dispersion of toner particles having a size in the range of about 0.1 to 2.0 microns, with typical toner particle diameters in the range 0.1 to 0.5 microns. In some embodiments, one or more charge control agents can be added after mixing, if desired.

Several characteristics of liquid toner compositions are important to provide high quality images. Toner particle size and charge characteristics are especially important to form high quality images with good resolution. Further, rapid self-fixing of the toner particles is an important requirement for some liquid electrophotographic printing applications, e.g., to avoid printing defects (such as smearing or trailing-edge tailing) and incomplete transfer in high-speed printing. Another important consideration in formulating a liquid toner composition relates to the durability and archivability of the image on the final receptor. Erasure resistance, e.g., resistance to removal or damage of the toned image by abrasion, particularly by abrasion from natural or synthetic rubber erasers commonly used to remove extraneous pencil or pen markings, is a desirable characteristic of liquid toner particles.

Another important consideration in formulating a liquid toner is the tack of the image on the final receptor. It is desirable for the image on the final receptor to be essentially tack-free over a fairly wide range of temperatures. If the image has a residual tack, then the image can become embossed or picked off when placed in contact with another surface (also referred to as blocking). This is particularly a problem when printed sheets are placed in a stack. Resistance of the image on the final image receptor to damage by blocking to the receptor (or to other toned surfaces) is another desirable characteristic of liquid toner particles.

To address some of these concerns, a film laminate or protective layer may be placed over the surface of the image. This laminate often acts to increase the effective dot gain of the image, thereby interfering with the accuracy of the color rendition of a color composite. In addition, lamination of a protective layer over a final image surface adds both extra cost of materials and extra process steps to apply the protective layer, and may be unacceptable for certain printing applications (e.g., plain paper copying or printing).

Various methods have been used to address the drawbacks caused by lamination. For example, approaches have employed radiation or catalytic curing methods to cure or crosslink the liquid toner after the development step in order to eliminate tack. Such curing processes are generally too slow for use in high speed printing processes. In addition, such curing methods can add significantly to the expense of the printing process. The curable liquid toners frequently exhibit poor self stability and crosslinking can result in brittleness of the printed ink.

Another method to improve the durability of liquid toned images and address the drawbacks of lamination is described in U.S. Pat. No. 6,103,781. This Patent describes a liquid ink composition containing organosols having side-chain or main-chain of crystallizable polymeric moieties. At column 6, lines 53-60, the authors describe a binder resin that is an amphipathic copolymer dispersed in a liquid carrier (also known as an organosol) that includes a high molecular weight (co)polymeric steric stabilizer covalently bonded to an insoluble, thermoplastic (co)polymeric core. The steric stabilizer includes a crystallizable polymeric moiety that is capable of independently and reversibly crystallizing at or above room temperature (22° C.). According to the authors, superior stability of the dispersed toner particles with respect to aggregation is obtained when at least one of the polymers or copolymers (denoted as the stabilizer) is an amphipathic substance containing at least one oligomeric or polymeric component having a weight-average molecular weight of at least 5,000 which is solvated by the liquid carrier. In other words, the selected stabilizer, if present as an independent molecule, would have some finite solubility in the liquid carrier. Generally, this requirement is met if the absolute difference in Hildebrand solubility parameters between the steric stabilizer and the solvent is less than or equal to 3.0 MPa^{1/2}.

As described in U.S. Pat. No. 6,103,781, the composition of the insoluble resin core is preferentially manipulated such that the organosol exhibits an effective glass transition temperature (Tg) of less than 22° C., more preferably less than 6° C. Controlling the glass transition temperature allows one to formulate an ink composition containing the resin as a major component so that the ink will undergo rapid film formation (rapid self-fixing) in liquid electrophotographic printing or imaging processes using offset transfer processes carried out at temperatures greater than the core Tg, preferably at or above 22° C. (Column 10, lines 36-46). The presence of the crystallizable polymeric moiety that is capable of independently and reversibly crystallizing at or above room temperature (22° C.) acts to protect the soft, tacky, low T_{g} insoluble resin core after fusing to the final image receptor. This acts to improve the blocking problem and erasure resistance of the fused, toned image at temperatures up to the crystallization temperature (melting point) of the crystallizable polymeric moiety.

In attempting to address tack of the image on a final receptor, one must also consider film strength and image integrity. As described in U.S. Pat. No. 6,103,781, for liquid electrophotographic toners (particularly liquid toners developed for use in offset transfer processes), the composition of the insoluble resin core is preferentially manipulated such that the organosol exhibits an effective glass transition temperature (Tg) of less than 22° C., and more preferably less than 6° C. Controlling the glass transition temperature allows one to formulate an ink composition containing the resin as a major component so that it will undergo rapid film formation (rapid self-fixing) in printing or imaging processes carried out at temperatures at least the core T_{g}, preferably at or above 22° C. (Column 10, lines 36-46).

As can be seen from the preceding, liquid toners are inherently more complex than dry toners to formulate. After each iteration or formulation, the toners must be tested, or screened, to see how the changes affect actual printing and how well the changed toner will work in an actual printing device. When an electrophotographic system uses dry toner, the measurements of various toner properties can be taken (with multiple testers) and a direct correlation can be inferred to indicate if the toner will perform satisfactorily or not. In liquid electrophotography, the number and interrelationship of the variables is extremely complex. As a result, the current liquid toner screening processes require labor-intensive and time-intensive printing of each liquid toner to be tested on a prototype printing device to determine whether or not a toner will be satisfactory.

### SUMMARY OF THE INVENTION

This invention addresses these and other problems associated with liquid toner screening. A first aspect of the invention is a liquid toner screening apparatus that will allow a drop of toner to be applied to a surface, plated to an electrically resistive roller, and transferred to a final substrate.

One element of the apparatus is a rigid planar platen having a top planar surface and a bottom planar surface. The platen is constructed so that the top and bottom planar surfaces are substantially horizontal. The rigid planar platen may be constructed of suitable materials, particularly composite materials, polymeric materials, ceramic materials, and metal or metal coated substrates, for example, polished or treated aluminum. The platen, or a top material layer, is electrically resistive and is connected to an electric power supply or to ground.

An electrically resistive compliant roller is another element of the apparatus, the resistive roller situated so that the circumference of the compliant roller may come into firm moveable contact with the final substrate (top planar surface) on the planar platen. The electrically resistive compliant roller preferably has a hardness of between 20-50 Shore A, but a preferred range is between 30-40 Shore A. In one embodiment, the electrically resistive compliant roller has at least two layers, namely an inner layer and an outer layer. If a two-layer embodiment is used, it is preferred that the inner layer has a resistivity between 10⁴ and 10⁸ ohm-cm and that the outer layer has a resistivity between 10⁸ and 10¹⁷ ohm-cm. Whether a single-layer or multi-layer construction is used, however, it is preferred that the total resistivity of the electrically resistive compliant roller is between 10⁵ and 10⁸ ohm-cm.

Movement of the electrically resistive compliant roller is enabled by a support element (frame, axle, rod, supported axle, support rollers, etc.) on which the rigid planar platen may be moveably mounted to enable a fixed range of horizontal motion. The rigid planar platen may support or depress the electrically resistive compliant roller such that the roller is still free to roll around its axis circumferentially along the platen. One embodiment may include a motor to propel the platen along its horizontal path. In another embodiment, the motor may be programmable to start and stop automatically as needed or in time with other events. A manually directed movement of the platen (with controls on the pressure of the roller against the platen) may also be used.

One element of the apparatus is a power supply electrically connected to the electrically resistive compliant roller and capable of providing a DC voltage to the electrically resistive compliant roller. In another embodiment, the power supply applies a DC and AC voltage. The support frame of the apparatus is preferably treated so as to neither impede nor enhance the electrical flow from the power supply to the electrically resistive compliant roller. In another embodiment, the power supply is programmable or controlled to automatically apply specified voltages at user specified times.

The apparatus of the invention may additionally have a final image-accepting substrate element placed on the platen, the final substrate being paper. In another embodiment, the final substrate may be an overhead projector film (OHP) or projection slide.

A second aspect of the invention is a method of screening liquid toner comprising the steps of: providing a screening apparatus such as the one described in the first aspect of the invention, with an electrically resistive compliant roller, a power supply for biasing the roller, a translating platen upon which the compliant roller can revolve and progress (the platen being either biased or electrically connected to ground), and an optional frame to support or resist the components or their movement; placing a drop of liquid toner on the platen in the prescribed horizontal path of the rotating compliant roller at least 1 inch in front of the roller and at least twice the length of the circumference of the compliant roller from the endpoint; supplying a DC voltage to the electrically resistive compliant roller; causing the platen and the charged electrically resistive compliant roller to move along the prescribed horizontal path; picking up (plating) the liquid toner with the charged electrically resistive compliant roller on a revolution across the platen, creating a plated toner oval; switching the bias on the electrically resistive compliant roller to an opposite polarity within the same revolution of the compliant roller; continuing the movement of the platen and electrically resistive compliant roller along the prescribed path; depositing the plated toner oval on the final substrate or on the platen on a subsequent revolution of the electrically resistive compliant roller, creating a resulting image; performing a mathematical analysis to determine if the resulting image on the substrate (final or platen substrate) is indicative of satisfactory or unsatisfactory performing toner.

In one embodiment of the second aspect of the invention, a final substrate medium having two dimensions (e.g., length and width, with depth being insignificant) may be positioned immediately prior to the endpoint of the platen such that there is at least the distance of the circumference of the electrically resistive compliant roller between the point of contact of the electrically resistive compliant roller to the platen and the nearest edge of the final substrate, and such that the length of the final substrate medium is at least two times the circumference of the electrically resistive compliant roller and such that the final substrate is placed on the platen length-wise with respect to the electrically resistive compliant roller.

### BRIEF DESCRIPTION OF THE FIGURES

FIG. 1 shows a simplified perspective view of elements of a screening apparatus that may be used in practicing a method according to the present invention.

FIG. 2 shows a perspective view schematic of a screening apparatus of the invention.

FIG. 3 shows a line drawing of an oval-shaped image obtained after the plating and transfer steps of the method.

FIG. 4 shows actual scanned images created from using the method and apparatus.

FIG. 5 shows a graphic representation of the correlation of multiple inks/toners between the screening apparatus and a prototype printing device.

FIG. 6 shows a graphic representation of the correlation between the percentage solids left in liquid toner (the vertical “X” axis) and the maximum achievable optical density (the horizontal “Y” axis).

### DETAILED DESCRIPTION OF THE INVENTION

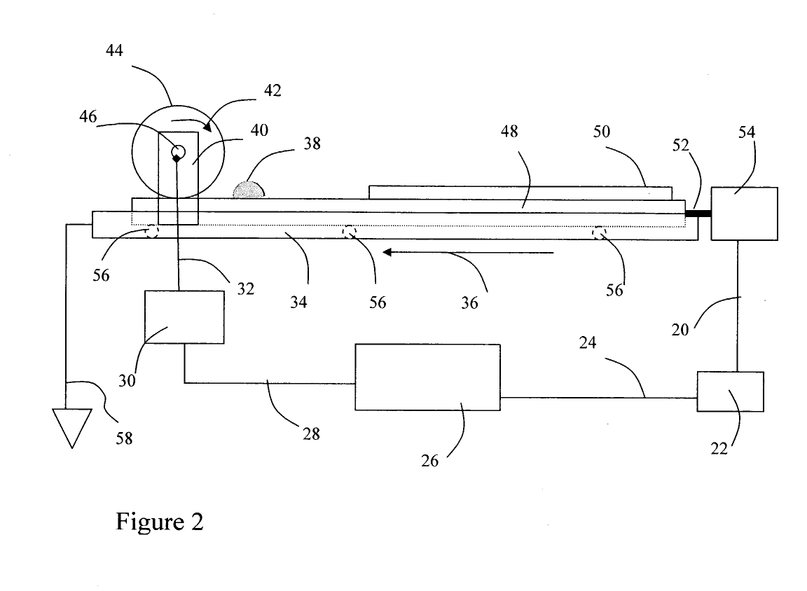

The apparatus of the invention may take many forms. Shown in FIG. 2 is one embodiment of the apparatus. It would be known and expected to one skilled in the art that certain elements within the apparatus are interchangeable or replaceable with equivalent materials and components and that alternative materials and components can be used. This apparatus is distinguished from electrostatic imaging systems in the prior art by its use of only one drop of liquid toner per test, and the application of that single drop in a non-imagewise manner. There is also no reservoir or imaging system. The two primary elements of the apparatus are the electrically resistive compliant roller (also herein called the “compliant roller”) 44 and the rigid platen 48.

The compliant roller 44 may be any size, but the inventors have found that a compliant roller 44 with a circumference of between 6-30 cm works best for making a small, but useful test device. The compliant roller 44 may be constructed out of a single material having a single set of electrical properties, or multiple materials or layers or multiple materials having many different electrical properties. It is also possible to have the composition of the platen 48 or the final substrate 50 graded in composition to accentuate toner/ink properties along a direction (e.g., 36) horizontal or perpendicular to the movement path of the platen 48 or roller 44. The actual number of layers used to form the compliant roller 44 is not important if the total resistivity for the roller is approximately between 10⁵ and 10⁸ ohm-cm. A general acceptable range of compliance or hardness would vary from about 20-50 Shore A, with a preferred hardness of between 30-40 Shore A. Various rubbers are known to have the electrical and physical requirements necessary to form the compliant roller 44 but many other materials could be used, such as elastomers, composites, layered materials, polymer coated materials, polymer saturated papers, foams, and the like. The use of higher or lower hardness surfaces may distort ink spreading in ways that diminish the quality or consistency of results. Those harder and softer materials may be used, but with that precaution.

The rigid planar platen 48 may be of any size and constructed out of a variety of materials (e.g., metals, metal oxides, metal coated substrates, metal oxide coated substrates, ceramic substrates, reinforced substrates, polymeric substrates and the like), but must be rigid enough to support the force/weight of the compliant roller 44 pressing downward on or at least resting on the planar surface. The planar surface may be grounded (shown here as 58) or biased, but it should form a complete circuit with the power supply 30 and the compliant roller 44. If a non-conductive rigid platen 48 is used, the surface contacting the compliant roller should be coated or covered with electrically conductive material that may be grounded 58 or biased. The rigid platen 48 should also be resistant to adherence of liquid toner (oleophobic) and need not be inherently so, but may be treated or coated to be oleophobic.

A frame 40, 34, and bearings 56 may be used to help the compliant roller 44 and the rigid platen 48 work together. Because the compliant roller 44 must maintain intimate, moveable contact with the platen 48 and/or the final substrate 50 thereon, it is necessary to support the compliant roller 44 by its axis 46, which is typically by a rod or axle, such as a conductive metal rod, but which preferably may be any conductive rigid material. One skilled in the art would know to use the radius of the compliant roller 44 to determine the distance needed between the axis 46 of the compliant roller 44 and the platen 48. In this embodiment, the axis 46 extends through compliant roller support 40 to support the compliant roller 44, assuring intimate, consistent pressure (e.g., the pressure does not vary by more then 10% as the roller 44 progresses over the platen 48) contact with the platen 48. Depending on the size and weight of the compliant roller 44, it may be necessary for the compliant roller support 40 to either bear some of the compliant roller's 44 weight to avoid excessive force or to apply force by forcing the compliant roller 44 into more intimate contact with the platen 48.

The frame 40, 34, and bearings 56 may also be used to stabilize and mobilize the platen 48. That is, the platen 48 may also be independently moveable, alone or in conjunction with the movement of the roller 44. In this embodiment shown in FIG. 2, the platen 48 rides on bearings 56 along tracks 34 (the part of the platen 48 that is behind a track 34 is shown by dashed lines). The direction the platen 48 will move for testing is shown by arrow 36. The compliant roller 44 in intimate contact with the platen 48 will simultaneously rotate in the direction indicated by arrow 42 such that the surface velocity of the compliant roller 44 is approximately equal to the surface velocity of the rigid platen 48. There are many means of ensuring smooth horizontal platen 48 movement, including for example the use of smooth operating stepping motors with lead screws, linear motors, pneumatic motors, magnetic drives, stabilizing systems, multiple bearing supports, air bearing supports and the like.

In one aspect of the invention, it is necessary that the compliant roller 44 be electrically biased. A power supply 30 may be electrically connected 32 to the compliant roller 44 by contacting the conductive axis 46. It would be known to one skilled in the art that the power supply 30 may be operated manually or automatically and may additionally include a controller, timer, and/or software (not shown specifically, but generally represented by box 26 connected electrically 28 to the power supply 30) to control the timing and application of the various biasing voltages.

As a matter of convenience, the platen 48 may be motorized, as shown by a drive mechanism 52 and motor 54. One skilled in the art would additionally know that a motor may also be controlled manually or automatically and may additionally include a controller, timer, and/or software (not shown specifically, but generally represented by box 22 connected electrically 20 to the motor 54) to control the timing and direction of the platen 48 movement. It is preferred that the rigid platen 48 and the compliant roller 44 travel at a speed of approximately 3 inches (7.5 cm)/sec, but a range of 2-10 inches/second (5.1-25.4 cm/sec) would be reasonable.

Finally, it is possible to electrically connect 24 motor 54 functions and automatic power supply 30 functions to one controller unit 26 (not discussed specifically, but shown generally as 26). The controller unit could coordinate such things as voltage changes and the direction and operation of the motor.

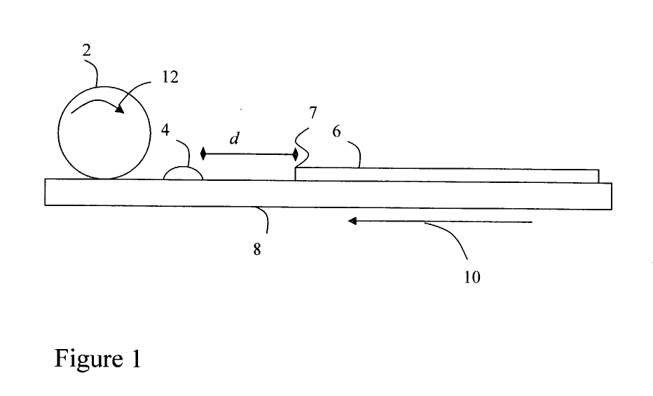

The method of using the invention is most simply explained in FIG. 1, where a biased compliant roller 2, rests in intimate contact with and at one end of a conductive, grounded or biased (not shown) platen 8 and a final substrate 6 rests on the opposite end of the platen 8. The final substrate 6 may be removable and/or disposable (such as paper or a thin polymeric film), or may simply be the biased platen 8 or a biased substrate (such as aluminum) residing thereon.

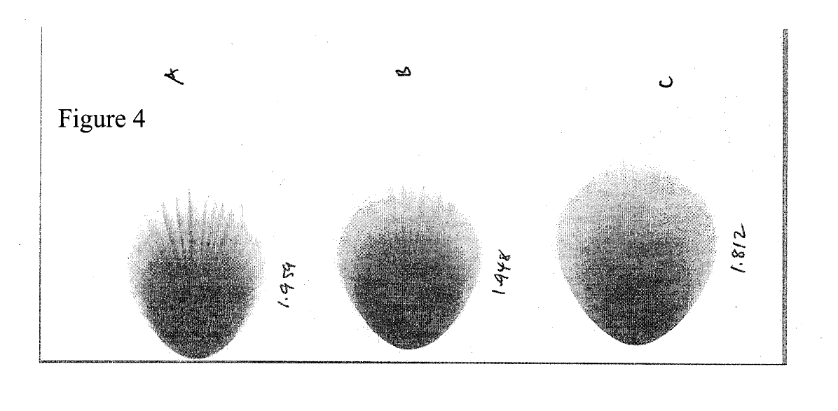

A drop of toner 4 is placed on the platen 8 between the biased compliant roller 2 and the final substrate 6. The volume of a drop is 0.0166 cm³ (±0.0016 cm³), or between about 0.01 and 0.025 cm³, and its weight may be about 0.015 g to 0.09 g. If the toner 4 is positively charged, the roller 2 must be negatively biased to pick up, or “plate” the toner on itself. In this embodiment of the invention, the platen 8 is moveable horizontally, as shown by arrow 10. Accordingly, the compliant roller 2 will simultaneously move in the direction indicated by arrow 12. The apparatus shown is for demonstrative purposes only, is non-limiting, and the platen 8 and/or compliant roller 2 may be propelled by any means, including but not limited to, manual movement or the use of a programmable stepper motor. The steps of the method include plating a drop of liquid toner 4 on the compliant roll 2 that is biased to attract the charged toner particles. Once the toner 4 has plated to the compliant roller 2, the bias to the roller is changed to repel the toner particles (i.e. a positive toner will require a more negative charge to plate the toner and a strongly positive bias to repel the toner) so that they are “printed” to the final substrate 6. The resulting image is essentially an oval shape with typically an uneven (jagged) “halo” of spikes around one end of the oval. The toner 4 may come into contact with and be plated to the compliant roller 2 on any given revolution of the compliant roller 2, but it is preferred that the “printing” to the final substrate 6 be completed on the revolution immediately subsequent to the plating step. The plated toner image (as seen in FIG. 4) is generally 6-12 cm in length. It is preferable to have the whole image on the surface of the compliant roller 2 before transfer to the final substrate 6. Therefore, the circumference of the compliant roller 2 is preferably greater than 12 cm (or has a radius of at least 2 cm). The distance d should also then be at least 12 cm.

The toner particles may be fixed (e.g., via heat and/or pressure) to the final substrate 6 before measurements are taken. The following examples demonstrate how and where measurements are taken and how to correlate the results to predicting the function of liquid toner.

### EXAMPLES

Specification and Configuration

For the purposes of this testing, a compliant roller having a two-layer construction was used. The resistivity of the roller was determined using the following test method.

The roller to be tested is wrapped with 0.004 inch (0.02 mm) thick shim stock of conductive metal band cut to a width of 0.5 inches (0.13 cm). The wrapped metal band is then secured for testing by a metal clamp that is tightened properly so that the shim stock maintains consistent and firm contact with the roller surface (if it is too tight the metal shim stock is actually lifted off of the roller surface at some points). Electrical connections are then made to the roll core (metal) and to the metal clamp. A controlled voltage is then applied to the electrical contacts at the core. The current that flows through the roll from the roll core to the metal wrap is measured. Typical applied voltages are either 10 volts or 100 volts and typical current measurements are in micro-amps.

The inner layer (formed around a conductive metal axis or core) was approximately 6 mm thick and had a resistivity (as measured above) of 10⁶ ohm-cm. The outer layer (comprising a very thin dielectric layer over the inner layer) was measured to be about 3×10¹⁰ ohm/cm. The equivalent total resistance of the sum of the layers was found to be about 10⁸ ohm-cm. The compliant roller for this testing had a hardness of 34 Shore A and was manufactured as an experimental roller for Samsung Electronics by Bando Corp. (2-21, Isogami-dori, 2-chome, Chuo-ku, Kobe 651-0086, JAPAN or P.O. Box 10060, 2720 Pioneer Drive, Bowling Green Ky. 42102-4860, USA).

A testing device as described above in FIG. 2 was designed and fabricated. In the two examples listed below, the initial voltage applied to the compliant roller during the plating step was −250V. The final transfer voltage used to transfer the plated toner to the final substrate (in this case, paper) was +450V. There was a time delay of 1 second between the applied voltage changes. The values were selected to simulate the values likely to be used in a real printer.

The traveling rigid platen was grounded in these experiments and was about 8″ long. The platen and motor were set to travel at a speed of 3 inches/second (7.6 cm/sec.); acceleration was 10 inches/sec² (25.4 cm/sec²). It took the roll 0.3 seconds (0.45 inches, 1.1 cm)) to reach constant speed, so the useful range of the platen was approximately 7.1 inches (18 cm). Therefore, using an 8 inch (20.3 cm) platen, a range of speeds for the tester could be between 2-10 inches/second 5.1-25.4 cm/sec).

Measurement

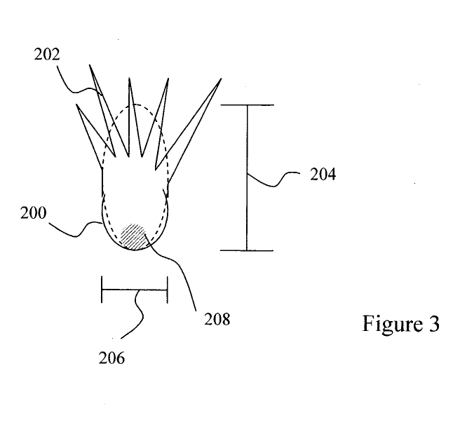

A representation of the oval-shaped image obtained after the plating and transfer steps of the method is shown in FIG. 3. (See FIG. 4 for some of the actual oval-shaped images). The oval-shaped image 200 may actually be an oval with sharp spikes 202 protruding from one end. The spikes 202 may be long or short and may be of uniform length or irregular length depending on the characteristics of the toner used. The area of the oval shaped image is estimated by measuring the width (w) 206 and the length (l) 204 of the spread. The width of the oval is taken at the widest part of the oval, just under where the spikes 202 meet the main portion of the image. The width scale 206 shows that measurement for the image in FIG. 3. The length of the oval is taken by measuring from the non-spike end of the oval, halfway up the spikes 202. The length scale 204 shows that measurement for FIG. 3. The dotted line in FIG. 3 shows the “oval” area that is being measured. The area of an oval (ellipse) is π/4wl and will be referred to hereinafter as “the area” (or “A”).

Assuming that the drop volume of various liquid toners having the same percentage of solids and dispersed in the same carrier liquid are the same, the thickness of the toner image should be proportional to l/A. l/A thus measured would then be proportional to the thickness of the toner layer deposited on the compliant roller in a real printer under a similar plating condition. Furthermore, if the plating and final transfers are nearly 100% efficient (utilizing voltages applied to the compliant roller and platen that have been optimized for nearly 100% transfer efficiency), l/A should be proportional to the optical density (OD) of a solid area printed with a real printer.

In some experiments, the optical density (OD) of the oval shaped or printed images were measured. For these tests, all measurements were taken with a Gretag® SPM 50 densitometer/color meter (made by Gretag, Inc.).

### Example 1

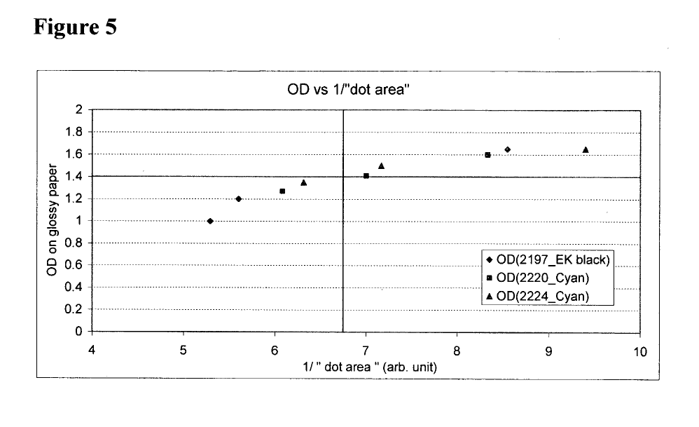

Three groups of toners having different chemical properties were used to test the validity of the device performance. Each toner was screened within the screening apparatus and subsequently screened in a real prototype printer configuration. FIG. 5 shows the correlation of each ink between the two devices. The vertical (Y) axis lists a range of optical densities that could be achieved on a prototype printer with the liquid toners. For this electrophotographic system, the optimum OD is approximately 1.4. The horizontal (X) axis shows possible values that could be achieved by using the toner screening apparatus as specified and configured above and determining l/A of each image.

For each experiment, the OD measurements of the toners printed on a prototype printer were taken as an average of at least three locations within the printed area.

As can be seen from the data in FIG. 5, toners with a l/A value of less than 6.75 tended to also print poorly (as evidenced by low optical densities). Toners with a l/A value (units may be arbitrary units of any area units such as 1/cm² or 1/m²) of greater than 6.75 printed to a density acceptable for this particular purpose. It is evident, however that there is a strong correlation between how well (to what density) a liquid toner prints and the l/A value obtained from using a liquid toner screening apparatus.

It was also observed, in general, that a toner that meets or exceeds printer specifications has a small spread with sharp, well-defined features, as seen in the image marked “A” in FIG. 4. An unsatisfactory toner tends to have a large spread with ill-defined features or no features all, as seen in the image marked “C” in FIG. 4.

### Example 2

The screening device may also be used to evaluate the performance of a satisfactory toner subjected to plating down, namely, the lowering of percentage of solids under extensive printing. A single cyan liquid toner was diluted to various solids percentages for the screening device evaluation. The term “percentage solids” refers to the ratio of solid toner particles to liquid carrier. It is determined by weighing a quantity of liquid toner, drying the carrier from the solid portion and re-weighing the solid portion, The second weight divided by the first weight is the “percentage solids.”

The OD of the plated and transferred ovals corresponds well with the OD results from images printed with a real printer. A few precautionary steps were taken when measuring OD for the ovals.

To avoid the complication of toner sedimentation between the time the toner drop is laid on the platform and the time the plating is initiated, the OD is measured at an area just outside the circular spread of the drop before the compliant roller passes over. In the experiments, a clear, circular, higher density area near the non-finger end of the oval image is visible. In FIG. 3, this area is indicated by a shaded region 208 indicating higher density at the site of the original drop placement. The OD measured outside of the initial drop placement region (also called “the drop footprint”) 208 is expected to be inversely proportional to the area of the oval.

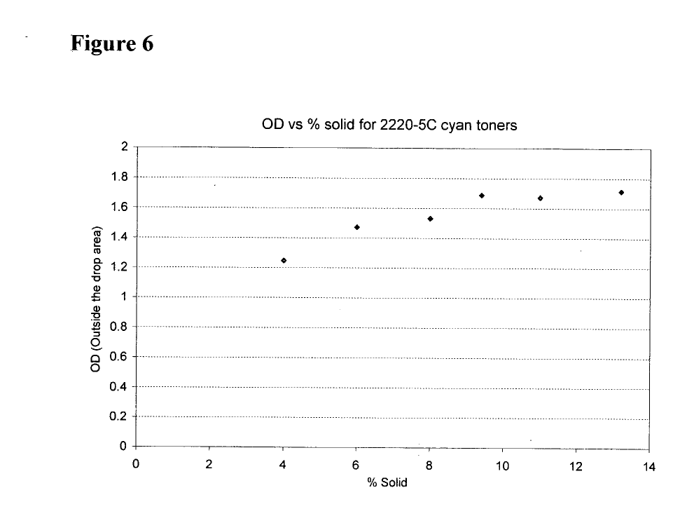

FIG. 6 shows the correlation between the percentage solids left in liquid toner (the vertical “X” axis) and the maximum achievable optical density (the horizontal “Y” axis). From this data, a user can determine that for this particular chemical composition of liquid toner, the toner will not fail to meet optical density standards until it falls below 6% solids (or has greater than 94% carrier liquid).

Other variations and modifications of this system are of course able to be combined with the underlying invention. For example, a separate roller or linear probe may be applied to the surface of the compliant roller (continuously or on command) to measure variations in the surface of the compliant roller, variations in the axial alignment of the compliant roller, optical measurements of the surface to measure these properties or to indicate surface roughness or changes in surface roughness, and then indicate when the roller should be changed or adjusted because of these measurements.

Look-up tables may be provided to assess the quality of the drop spreading characteristics. These may be electronic look-up tables where the comparison is made with and evaluated from scanned, analog or digital image data compared to the look-up table, or a manual (visual) look-up table in which a trained observer compares specific parameters of the spread drop or generally compares images of the spread drop to images or characteristics in a visual look-up table.

The look-up tables may identify specific properties of the ink that are shown to be deficient because of the nature of observed or measured properties of the spread spot. It is even possible that a blade applicator or squeegee-type applicator (with the appropriate electrical properties) could be used in place of the roller.

What is claimed is

1. A screening apparatus for evaluating the electrostatic imaging properties of a liquid toner comprising:
a planar platen having a top planar surface and a bottom planar surface, the platen situated so that the top planar surface and the bottom planar surface are substantially horizontal, the planar platen being electrically connected to an electrical power supply or to ground;
an electrically resistive compliant roller having a circumference, the circumference of the electrically resistive compliant roller positioned in or moveable into firm moveable contact with the planar platen;
a support frame on which the planar platen is mounted to enable a range of horizontal motion relative to the electrically resistive compliant roller, and which support frame may support or depress the electrically resistive compliant roller such that the roller remains free to rotate about its axis as the compliant roller relatively moves along the platen;
a power supply electrically connected to the electrically resistive compliant roller that provides a DC voltage to the electrically resistive compliant roller; and
the support frame neither impeding nor enhancing the current flow from the power supply to the electrically resistive compliant roller.

2. The apparatus of claim 1 wherein the planar platen comprises a rigid planar platen of polished aluminum.

3. The apparatus of claim 1 wherein the planar platen comprises a rigid material having attached or mounted thereto a conductive surface.

4. The apparatus of claim 1 wherein a final image retaining substrate is placed on the top platen surface.

5. The apparatus of claim 4 wherein the image retaining substrate present on the platen comprises paper.

6. The apparatus of claim 4 wherein the image retaining substrate comprises a polymeric film having a thickness less than 75 microns as the image retaining substrate on the platen.

7. The apparatus of claim 4 wherein the image retaining substrate comprises a conductive material, such as aluminum.

8. The apparatus of claim 1 wherein the electrically resistive compliant roller has a hardness of between 20-50 Shore A durometer hardness.

9. The apparatus of claim 1 wherein the electrically resistive compliant roller has at least two layers, an inner layer and an outer layer.

10. The apparatus of claim 9 wherein the inner layer has a resistivity between 10⁴ and 10⁸ ohm-cm.

11. The apparatus of claim 9 wherein the outer layer has a resistivity between 10⁸ and 10¹⁴ ohm-cm.

12. The apparatus of claim 9 wherein the total resistivity of the electrically resistive compliant roller is between 10⁵ and 10⁸ ohm-cm.

13. The apparatus of claim 1 wherein the total resistivity of the electrically resistive compliant roller is between 10⁵ and 10⁸ ohm-cm.

14. The apparatus of claim 1 further comprising a motor to propel the platen along a horizontal path.

15. The apparatus of claim 14 wherein the motor is programmable to automatically move upon activation of a switch.

16. The apparatus of claim 1 wherein the power supply additionally supplies an AC voltage to the DC voltage being used for the test.

17. The apparatus of claim 1 wherein the power supply is programmable or controlled to automatically apply used specified voltages at user specified times.

18. A method of screening a liquid toner comprising the steps of:
providing a screening apparatus having:
a) a planar platen having a top planar surface and a bottom planar surface, the top planar surface and the bottom planar surface of the platen being substantially horizontal;
b) an optional receiving substrate that may be removably placed on the top planar surface;
c) an electrically resistive compliant roller having a circumference positioned in or moveable into contact with the receiving substrate or the planar platen;
d) a support frame on which the planar platen may be mounted for movement relative to the compliant roller to enable a range of horizontal motion relative to the compliant roller, and which planar platen may support or depress the electrically resistive compliant roller such that the roller maintains its ability to rotate around its axis when the compliant roller moves relative to the final substrate or the platen;
e) a power supply electrically connected to the electrically resistive compliant roller and capable of providing a DC voltage to the electrically resistive compliant roller; and
f) the support frame neither impeding nor enhancing current flow from the power supply to the electrically resistive compliant roller;
positioning the planar platen so that the electrically resistive compliant roller rests on a first end of the top platen surface that allows the planar platen to relatively move horizontally with respect to an axis of the electrically resistive compliant roller while the electrically resistive compliant roller rotates along its axis and the circumference of the electrically resistive compliant roller maintains contact with the platen;
placing a drop of liquid toner on the platen in a prescribed horizontal path of the electrically resistive compliant roller at least 1 inch in front of the roller and at least twice the length of the circumference of the compliant roller from an anticipated endpoint in movement of the electrically resistive compliant roller with respect to the planar platen;
supplying a DC voltage to the electrically resistive compliant roller to provide a charged electrically resistive compliant roller;
causing the platen and the charged electrically resistive compliant roller to move along the prescribed horizontal path;
plating the drop of liquid toner onto the charged electrically resistive compliant roller on an initial revolution relative to the platen, creating a plated toner shape;
switching the bias on the charged electrically resistive compliant roller to an opposite polarity within the initial revolution;
continuing the relative movement of the platen and electrically resistive compliant roller along the prescribed path;
depositing the plated toner shape on a final substrate or on the platen on a subsequent revolution of the electrically resistive compliant roller, creating a resulting image; and
comparing properties of the resulting image to a standard or look-up table.

19. The method of claim 18 where an AC voltage is added to the DC voltage.

20. The method of claim 18 wherein the comparing properties is used to indicate satisfactory or unsatisfactory performance of the liquid toner.

21. The method of claim 18 wherein the comparison comprises a visual comparison.

22. The method of claim 18 wherein the comparison comprises a mathematical comparison.

23. The method of claim 18 wherein a final receiving substrate medium having dimensions of at least length and width is positioned immediately prior to the anticipated endpoint such that there is at least the distance of the circumference of the electrically resistive compliant roller between the point of contact of the electrically resistive compliant roller to the platen and the nearest edge of the final substrate, and such that the length of the final substrate medium is at least two times the circumference of the electrically resistive compliant roller and such that the final substrate is placed on the platen length-wise with respect to the electrically resistive compliant roller.
